(19) United States
(12) Patent Application Publication
(10) Pub. No.: US 2007/0141625 A1
(43) Pub. Date: Jun. 21, 2007

(54) Method for assessing risk of and predisposition to development of a pathology related to the presence of anti-epcr autoantibodies

(76) Inventors: Jose Hermida Santos, Pamplona (ES); Ramon Montes Diaz, Pamplona (ES); Veronica Hurtado Linares, Burgos (ES)

Correspondence Address: MOORE & VAN ALLEN PLLC, P.O. BOX 13706, Research Triangle Park, NC, 27709, US

(21) Appl. No.: 10/588,740

(22) PCT Filed: Feb. 3, 2005
(86) PCT No.: PCT/ES05/00046

§ 371 Date: Oct. 20, 2006

(51) Int. Cl. G01N 33/53

(52) U.S. Cl. 435/7.1

(57) ABSTRACT

The present invention relates to a method for detecting the presence of high levels of autoantibodies against protein C/activated protein C endothelial receptor (EPCR). The invention is characterised in that it comprises in vitro detection and quantification of anti-EPCR autoantibodies in a sample.

### FIELD OF THE INVENTION

The present invention relates to a method for detecting high levels of autoantibodies against endothelial protein C/activated protein C receptor (EPCR) in a sample, by its detection and in vitro quantification.

### BACKGROUND OF THE INVENTION

Autoimmune Diseases

Autoimmune diseases are characterized by the presence of immune reactions in which something induces an immune reaction against the host tissues, and the production of abnormal antibodies that attack such tissues (autoantibodies). These autoimmune diseases include disorders such as antiphospholipid syndrome (APLS), rheumatoid arthritis, systemic lupus erythematosus, autoimmune vasculitis in general, etc.

APLS is characterized by vascular thrombosis (venous, arterial or microvascular) and complications during pregnancy (fetal death, premature birth or multiple spontaneous miscarriage) associated with the presence of antiphospholipid antibodies. These antibodies are heterogeneous and recognize a variety of combinations of phospholipids, phospholipid binding proteins, or both. The most commonly detected antiphospholipid antibody subgroups comprise the so-called anticoagulant lupus antibodies (ACL), anticardiolipin antibodies and antiglycoprotein I β2 antibodies. Other antiphospholipid antibodies not included in the classical laboratory criteria are presently being investigated. Such antibodies are targeted to phospholipids other than cardiolipin, such as phosphatidylethanolamine, or to phospholipid binding proteins such as annexin V and protein S. However, little is known about the mechanisms relating the presence of antiphospholipid antibodies to vascular thrombosis and miscarriage.

Vascular Diseases

Vascular diseases are of three principal types depending on the kind of the vessel involved (arterial, venous or small-caliber vessels of the microcirculation). In the case of arterial vascular diseases, parietal sclerosis reduces blood flow through the vessel lumen, and therefore chronically reduces blood supply to the territories irrigated by the damaged blood vessel. This atherosclerotic lesion can suffer complications and give rise to thrombus formation within the artery—totally occluding the latter and thus obstructing blood flow entirely. In this case, tissue infarction results. The most frequent examples of this phenomenon are myocardial infarction, when thrombosis affects a coronary artery, or stroke—when the affected vessel is a brain artery. In the case of venous vascular disease, thrombosis complicates return blood flow to the heart. When a fragment of the thrombus in the thrombotic venous wall becomes detached, it will migrate within the bloodstream until it becomes lodged in the pulmonary venous circulatory circuit—giving rise to acute pulmonary failure (a situation known as pulmonary embolism). Diseases of the microcirculation develop secondary to inflammation and/or thrombosis of the vessels of the microcirculation in different organs, and manifest as organ failure which microcirculation is damaged. Vascular diseases are an important cause of morbidity and mortality in western countries. Specifically, according to data from the Instituto Nacional de Estadística (INE) (Spanish National Institute of Statistics) corresponding to the year 2000, cardiovascular diseases are the first cause of death in Spain (representing approximately 35.0% of total mortality). Among the most frequent cardiovascular disorders, vascular or thrombotic arterial diseases of the heart (mainly acute myocardial infarction) constitute the first cause of death. At present, a number of molecular risk factors have been identified that can account for the appearance of thrombosis in some patients. One such risk factor is the presence of so-called antiphospholipid antibodies. It was originally believed that these autoantibodies were targeted to anionic phospholipids; however, subsequently it has been shown that many of these autoantibodies are targeted to complexes formed between proteins such as glycoprotein I β2 or prothrombin, and phospholipids. More recently, other proteins with anticoagulant roles have also been implicated, such as protein C (PC), protein S, thrombomodulin or annexin V—thus explaining why the presence of these autoantibodies predisposes to thrombosis.

Obstetric Complications

Obstetric complications fundamentally comprise fetal death after the tenth week of pregnancy, the birth of premature infants, spontaneous miscarriage before the tenth week of pregnancy, delayed intrauterine growth, eclampsia and pre-eclampsia.

EPCR

Activated protein C (APC) is one of the principal coagulation cascade regulatory proteins. PC, the zymogen of APC, is activated by thrombin bound to thrombomodulin on the surface of the endothelial cells. APC, in combination with protein S (its non-enzymatic cofactor), exerts its anticoagulant role via the proteolysis of activated factors V and VIII. Genetic and acquired defects in thrombomodulin, PC and protein S have been detected in patients with venous and/or arterial thrombosis. Endothelial PC/activated PC receptor (EPCR) is a glycoprotein expressed on the membrane of endothelial cells that specifically and with high affinity binds PC and APC. In order for EPCR to be functional, it must be bound to a phospholipid molecule that stabilizes its three-dimensional structure. The binding of PC to EPCR markedly increases its activation by the thrombin-thrombomodulin complex on the endothelial cell surface. The mission of EPCR is to concentrate PC on the endothelial surface and present it to the thrombin-thrombomodulin complex—thereby favoring efficient PC activation. EPCR induces an approximately 9-fold increase in the PC activation index on the surface of endothelial cells in vivo—as a result of which it is responsible for 90% of the circulating levels of APC. Moreover, only when APC is bound to EPCR can it activate protease-activated receptor-1, that generates a “cytoprotective” cell signal and blocks apoptosis.

EPCR is mainly expressed by the endothelium of veins and arteries, particularly those of large and medium caliber. Moreover, it is intensely expressed by the syncytiotrophoblast. In these locations EPCR prevents thrombosis and favors good cell function both of the endothelium and the syncytiotrophoblast. There is increasingly solid evidence to suggest that EPCR plays a role in the maintenance of pregnancy, since deletion of the EPCR gene in knock out mice causes placental thrombosis and early embryonic death in these mice.

### SUMMARY OF THE INVENTION

The present invention refers to a method for the determination of anti-EPCR autoantibodies (IgG, IgA and IgM) in a sample from a subject. On the other hand, it has been demonstrated that these autoantibodies are present in patients diagnosed with autoimmune diseases (APLS and disseminated lupus erythematosus), in patients with vascular diseases (venous and arterial thrombosis) and in female patients with obstetric complications. The examples that accompany the present description illustrate, among other things, the fact that the presence of anti-EPCR autoantibodies in serum or plasma is increased in patients with autoimmune diseases (determined in patients with APLS or disseminated lupus erythematosus), in patients with vascular diseases, such as arterial thrombosis, for example, myocardium infarction (determined both in patients with APLS and in patients without APLS), or ischemic stroke (determined in patients with APLS), or venous thrombosis (determined in patients with APLS), as well as in patients with obstetric complications, such as fetal death (determined both in women with APLS and in women without APLS), or multiple miscarriage (determined in patients with APLS).

The authors of the present invention have discovered that the presence of anti-EPCR autoantibodies in serum or plasma from patients with autoimmune diseases, and/or patients with vascular diseases and/or in patients with obstetric complications, is increased when compared with samples from healthy subjects, not affected by such diseases. These evidences make said anti-EPCR autoantibodies a useful marker for in vitro evaluation of the risk and susceptibility of a subject to develop a disease associated with the presence of increased levels of autoantibodies against EPCR, such as an autoimmune disease, a vascular disease, or obstetric complications.

Studies have been made of the presence of anti-EPCR autoantibodies in patients with APLS and their relation to fetal death. An evaluation has also been made of the effect of these autoantibodies upon the generation of APC on the endothelial surface. Afterwards, a study has been made of the association of anti-EPCR autoantibodies to fetal death in a paired case-control study. The results obtained support the notion that anti-EPCR autoantibodies constitute a risk factor for fetal death. Prevention of the activation of PC on the cell surfaces that express EPCR could be one mechanism by which these autoantibodies exert their pathological effects.

### BRIEF DESCRIPTION OF THE FIGURES

FIG. 1 shows the expression of rhsEPCR in Pichia pastoris. The rhsEPCR was purified from the supernatant of stably transformed P. pastoris cells, as described in the section relating to Materials and Methods (see Example). Ten μl of each of three fractions containing rhsEPCR were separated by SDS-PAGE, and the proteins were detected using GELCODE Blue (A) or via Western blot with the monoclonal anti-myc antibody (Invitrogen) (B).

FIG. 2 compares the level of anti-EPCR autoantibodies in patients diagnosed with APLS and in controls. The levels of anti-EPCR autoantibodies are shown. Antibodies of IgM isotype: controls (median=45 AU, arbitrary units), patients (median=57 AU); antibodies of IgA isotype: controls (median=31 AU), patients (median=39 AU); and antibodies of IgG isotype: controls (median=72 AU), patients (median=75 AU).

FIG. 3 shows the effect of anti-EPCR autoantibodies on the generation of APC by endothelial cells, where the generation of APC in the presence of anti-EPCR autoantibody of isotype M can be seen in patient C, compared with the generation of APC in the absence of antibody and in the presence of non-inhibiting antibody. For each condition 2-4 independent experiments were performed.

### DETAILED DESCRIPTION OF THE INVENTION

Definitions

In order to facilitate understanding of the present patent application, the meanings of some terms and expressions used in the context of the invention are explained below.

The term “subject” refers to a member of a mammalian species, and includes but is not limited to domestic pets, primates and humans; the subject is preferentially a human (male or female) of any age or race.

The expression “autoimmune diseases” refers to those disorders in which the immune system reacts against the host tissues, giving rise to a broad range of disorders. For illustrative purposes, such diseases include (among other conditions) APLS, systemic lupus erythematosus, rheumatoid arthritis, autoimmune vasculitis, etc.

The expression “vascular diseases” refers to those disorders that affect the blood vessels. When an artery is involved, the subsidiary territory irrigated by the vessel suffers a lack of perfusion; this condition is usually secondary to arterial occlusion attributable to an atherosclerotic lesion in the wall or to thrombosis or both simultaneously. Venous involvement is in turn defined by the complication of blood return to the heart from the affected peripheral territory, and is usually the result of venous thrombus formation leading to vessel occlusion. When it affects to microcirculation it is characterized by the impairment of the organ whose microcirculation is affected for carrying out its function. As an example, such diseases include (among other disorders) arterial vascular disorders such as myocardial infarction, stroke, transient cerebrovascular accidents, ischemia of the limbs, atherosclerosis, aneurysms, etc., as well as venous vascular diseases such as superficial and deep venous thrombosis, pulmonary embolism, etc., and microcirculatory pathology (thrombosis) in the form of organ failure seen during infections or in the context of autoimmune diseases.

The expression “obstetric complications” refers to those disorders affecting the development of pregnancy, as relates to both the gestating mother and to embryo or fetus. Examples include miscarriage, fetal death, premature birth, delayed intrauterine growth, eclampsia and pre-eclampsia.

The term “autoantibody” refers to the antibodies produced by a subject and targeted to (or specific for) host structures and tissues of the own producing organism, such as, for example, antiplatelet autoantibodies, antithyroid autoantibodies, autoantibodies to erythrocytes, etc. In this sense, the term “anti-EPCR autoantibody” refers to immunoglobulins or antibodies produced by the subject and specifically targeted to EPCR of his or her own tissues.

The term “epitope”, as used in the present invention, refers to an antigenic determinant of a protein, such as the amino acid sequence thereof, which is recognized by the antibody in question.

The terms “peptide” and “polypeptide” refer to molecular chains of amino acids that represent a protein fragment. The terms “protein” and “peptide” are used indistinctly.

The present invention is based on the observation that the production of anti-EPCR autoantibodies in patients with autoimmune diseases, and/or in patients with vascular diseases and/or in patients with obstetric complications is increased in comparison to samples from healthy subjects without such diseases. This evidence defines such anti-EPCR autoantibodies as a useful marker for in vitro evaluation of the risk and susceptibility of a given subject to develop a pathology related to the presence of high levels of autoantibodies against EPCR.

As used in this description, the expression “high levels of anti-EPCR autoantibodies” refers to levels of AU (arbitrary units) equal to or in excess of percentile 50 in the normal population, including, for example, levels of AU equal to or in excess of percentile 60 in the normal population, equal to or in excess of percentile 70 in the normal population, equal to or in excess of percentile 80 in the normal population, equal to or in excess of percentile 90 in the normal population, and equal to or in excess of percentile 95 in the normal population. Due to inter-subject variability (e.g., aspects relating to race, etc.) it is very difficult (if not practically impossible) to establish absolute values indicative of high levels of anti-EPCR autoantibodies applicable to all subjects. Such percentiles can easily be calculated by means of a conventional procedure involving the testing of a group of normal subjects (i.e., people with no diagnosis of autoimmune disease, or antecedents of vascular disease or obstetric complication at the time of testing) of the levels of anti-EPCR autoantibodies. The determination of anti-EPCR autoantibodies can be done using any conventional method, for example the ELISA described under “Materials and Methods” (Example 1). Logically, each subject will present a certain level (AU) of anti-EPCR autoantibodies, and a concrete anti-EPCR autoantibody level will be identified above which 50% of the analyzed population is found. This value is the percentile 50. Obviously, a value (AU) also exists above which 40% of the normal subjects tested can be found—this value corresponding to percentile 60. In turn, other values can be defined above which 30%, 20%, 10% and 5% of the normal subjects tested can be found—corresponding to percentiles 70, 80, 90 and 95, respectively.

The invention provides a method for detecting the presence of high levels of autoantibodies against endothelial protein C/activated protein C receptor (EPCR) in a sample, characterized by comprising the in vitro quantification of autoantibodies against EPCR in said sample from a subject. These high levels of autoantibodies are related to a pathology selected from an autoimmune disease (for example, APLS, systemic lupus erythematosus, rheumatoid arthritis, autoimmune vasculitis, etc.); a vascular disease (for example, arterial vascular disease such as myocardial infarction, stroke, transient cerebrovascular accidents, ischemia of the limbs, atherosclerosis, aneurysms, thrombosis, etc., or venous vascular disease such as superficial or deep venous thrombosis, pulmonary embolism, etc., or microcirculatory vascular disease); and obstetric complications (for example, miscarriage, fetal death, premature birth, delayed intrauterine growth, eclampsia, pre-eclampsia, etc.) . Thus, the method the subject-matter of the present invention is applicable to the determination of the variation of the levels of anti-EPCR autoantibodies over a given time-span. Such determinations object of the instant invention are completed by their comparison to normal levels of anti-EPCR autoantibodies.

Said method comprises a step in which a sample is collected from the subject, such as a sample of serum or plasma, which can be obtained by any conventional method, e.g., blood collection.

The samples can be obtained from subjects with previously diagnosed or non-diagnosed autoimmune diseases or vascular disorders, or obstetric complications. They can also be obtained from subjects undergoing treatment, or that have been previously treated for such diseases or complications.

Given the nature of the method of the invention, the detection and quantification of these anti-EPCR autoantibodies is carried out by means of an immune test coupled to a marker that allows detection and quantification of the formation of specific antigen-antibody complexes, e.g., an immunochromatographic test (latex, colloidal gold, etc.), an immune test in which the marker is fluorescent, an isotope, a heavy metal, an enzyme, a luminescent marker, a chemiluminescent marker, a chromogen, etc.

A broad range of well known tests can be used in the present invention, involving the use of unlabeled antibodies (primary antibody) and labeled antibodies (secondary antibody). These techniques include Western-blot or Western transference, ELISA (enzyme-linked immunosorbent assay), RIA (radioimmunoassay), etc.

In a particular embodiment, the preferred immune test in the method of the invention, which allows the detection and/or quantification of these anti-EPCR autoantibodies is an ELISA test which comprises:
a) immobilizing in a solid support a polypeptide comprising the sequence of amino acids of EPCR or a fragment thereof containing at least one epitope that can be recognized by an anti-EPCR autoantibody;
b) incubating said immobilized polypeptide with a sample suspected to contain anti-EPCR autoantibodies, obtained from said subject, for sufficient time to allow binding of the antibodies to the immobilized polypeptide, and the formation of polypeptide-anti-EPCR autoantibody complexes;
c) removing the remaining sample not bound to the immobilized polypeptide;
d) incubating said polypeptide-anti-EPCR autoantibody complexes with a second antibody conjugated to an enzyme, where said second antibody is able to bind to said anti-EPCR autoantibodies.

This polypeptide comprising the EPCR amino acid sequence or a fragment thereof containing at least one epitope that can be recognized by an anti-EPCR autoantibody can be a polypeptide comprising the sequence of amino acids of full length EPCR, or a polypeptide comprising the sequence of amino acids of an EPCR fragment and containing at least one epitope capable of being recognized by an anti-EPCR antibody. In a particular embodiment, the mentioned polypeptide is a fusion protein comprising:
(i) a region A composed of a polypeptide containing the EPCR amino acid sequence or a fragment thereof containing at least one epitope capable of being recognized by an anti-EPCR antibody; and
(ii) a region B composed of a polypeptide containing an amino acid sequence of use for isolating or purifying the mentioned fusion protein, and/or a sequence of amino acids of use for anchoring the mentioned fusion protein to a solid support.

This region B can be bound to the amino terminal extreme of region A or to the carboxyl terminal extreme of region A.

In a particular embodiment, region A comprises the amino acid sequence of the soluble part of human EPCR.

Region B comprises an amino acid sequence of use for the isolation or purification of the previously defined fusion protein, and/or a sequence of amino acids of use for anchoring the mentioned fusion protein to a solid support. Practically any sequence of amino acids that can be used to isolate or purify a fusion protein (generically referred to as “tag” peptides) , and/or any sequence of amino acids capable of being used for anchoring a fusion protein to a solid support can be present in region B. Occasionally, the amino acid sequence of use for the isolation or purification of the fusion protein can also act as a sequence of amino acids of use for anchoring the mentioned fusion protein to a solid support, and vice versa. In a particular embodiment, region B comprises a sequence of amino acids of use for the isolation or purification of a fusion protein and a sequence of amino acids of use for anchoring a fusion protein to a solid support.

As an example, this sequence of amino acids of use in isolating or purifying a fusion protein and/or the sequence of amino acids of use in anchoring a fusion protein to a solid support can be Arg-tag, His-tag, FLAG-tag, Strep-tag, an epitope capable of being recognized by an antibody, such as c-myc-tag, SBP-tag, S-tag, calmodulin binding peptide, cellulose binding domain, chitin binding domain, glutathione S-transferase-tag, maltose binding protein, NusA, TrxA, DsbA, Avi-tag, etc. (Terpe K., Appl. Microbiol. Biotechnol. (2003), 60:523-525), a sequence of amino acids such as Ala-His-Gly-His-Arg-Pro (SEQ ID NO: 4) (2, 4, and 8 copies), Pro-Ile-His-Asp-His-Asp-His-Pro-His-Leu-Val-Ile-His-Ser (SEQ ID NO: 5), Gly-Met-Thr-Cys-X-X-Cys (SEQ ID NO: 6) (6 repetitions), β-galactosidase, VSV-glycoprotein (YTDIEMNRLGK), etc.

In a particular embodiment, said region B consists of a polypeptide comprising an epitope capable of being recognized by an antibody (such as the c-myc epitope, recognized by an anti-c-myc antibody), and a tail of histidines (His-tag).

In the Example accompanying this description, it is disclosed the production of a polypeptide, called rhsEPCR, consisting of a fusion protein comprising the amino acid sequence of the soluble part of human EPCR (hsEPCR), the amino acid sequence corresponding to c-myc epitope and a tail of histidines—the amino acid sequence being shown in SEQ ID NO: 3.

The polypeptide to be used in the method of the invention can be obtained by conventional methods, for example, by expression in an appropriate expression system.

The second antibody to be used in the previously mentioned ELISA test is an immunoglobulin isotype-specific antibody originated from a species different to that of the study subject, thereby allowing characterization of the isotype of the anti-EPCR autoantibodies. As an example, this second antibody specific of a given immunoglobulin isotype is selected from an anti-human IgG antibody, an anti-human IgM antibody, an anti-human IgA antibody, and their mixtures. In a particular embodiment, the second antibody is conjugated to a marker allowing detection of the complex, such as an enzyme (e.g., peroxidase, alkaline phosphatase, etc).

In another aspect, the invention supplies a method for assessing the risk and susceptibility of a subject to develop a pathology related to the presence of high levels of anti-EPCR autoantibodies in said subject, comprising in vitro quantification of autoantibodies against EPCR in a sample from said subject.

In a particular embodiment, the pathology related to the presence of high levels of autoantibodies against EPCR in a subject is selected from an autoimmune disease, such as APLS, systemic lupus erythemotosus, reumatoid arthritis, autoimmune vasculitis, etc.; a vascular disease, such as, an arterial vascular disease, e.g. myocardial infarction, stroke, transient cerebrovascular accidents, ischemia of limbs, atherosclerosis, aneurysms, thrombosis, etc., or a venous vascular disease, e.g. superficial o deep venous thrombosis, pulmonary embolism, etc. or microcirculatory vascular disease such as a microcirculatory thrombosis, organic failure occurring during infections or autoimmune disease, etc.; and an obstetric complication, for example, miscarriage, fetal death, premature birth, delayed intrauterine growth, eclampsia, pre-eclampsia, etc.

The method provided by the present invention is based on the fact that subjects diagnosed with an autoimmune or vascular disease o with obstetric complications, have high levels of anti-EPCR autoantibodies, when compared to the corresponding levels in subjects without a clinical history of such diseases or obstetric complications.

The method used to evaluate (assess) the risk and susceptibility of a subject to develop a pathology related to the presence of high levels of anti-EPCR autoantibodies provided by this invention is completed by comparing the levels of autoantibodies determined in the study subject sample with normal levels (defined as those found in a population of normal subjects such as that mentioned above in reference to definition of the expression “high levels”). Said method is based on immunological assays previously described in this section.

In another aspect, the invention is related to a method for in vitro monitorization of the effect of therapy administered to a subject presenting a pathology related to the presence of high levels of anti-EPCR autoantibodies, comprising the in vitro quantification of these anti-EPCR autoantibodies in a sample of the mentioned subject. The method is carried out as mentioned above, though in this case the samples originate from subjects previously diagnosed with some autoimmune or vascular disease, or presenting some obstetric complication, subjected to therapy. The method allows evaluation of the effect of therapy, i.e., its efficacy and effectiveness, applied to the subject undergoing treatment, with the purpose (for example) of either maintaining therapy or modifying it.

In another aspect, the invention is related to the use of anti-EPCR autoantibodies in a method to evaluate the presence of high levels of autoantibodies against EPCR in a sample from a subject. In a particular embodiment, said presence of high levels of anti-EPCR autoantibodies related to a pathology, is selected from an autoimmune disease, a vascular disease and obstetric complications. An increased level of anti-EPCR autoantibodies in the subject is associated with an increased risk or susceptibility to develop a pathology related with the presence of high levels of anti-EPCR autoantibodies, such as autoimmune disease, vascular disease and/or obstetric complications.

In another aspect, the invention is related to the use of a polypeptide comprising the sequence of amino acids of EPCR or a fragment thereof containing at least one epitope capable of being recognized by an anti-EPCR autoantibody, in a method for evaluating the presence of autoantibodies against the endothelial receptor EPCR in a sample. Said method comprises the detection and in vitro quantification of autoantibodies anti-EPCR in said sample. In a particular embodiment, this pathology related to the presence of high levels of anti-EPCR autoantibodies was selected from an autoimmune disease, a vascular disease and obstetric complications.

In a particular embodiment, the mentioned polypeptide comprising the EPCR amino acid sequence or a fragment thereof containing at least one epitope capable of being recognized by an anti-EPCR autoantibody is a polypeptide such as that previously defined on describing the ELISA test for detecting and/or quantifying anti-EPCR autoantibodies. In a particular embodiment, this polypeptide is the so-called rhsEPCR (see Example), consisting of a fusion protein comprising the sequence of amino acids of the soluble part of human EPCR (hsEPCR), the amino acid sequence corresponding to c-myc epitope and a tail of histidines—the sequence of which is shown in SEQ ID NO: 3.

In another aspect, the invention is related with a kit designed for in vitro evaluation of high levels of anti-EPCR autoantibodies, comprising a polypeptide with the EPCR amino acid sequence or a fragment thereof containing at least one epitope capable of being recognized by an anti-EPCR autoantibody. In a particular embodiment, this polypeptide comprising the EPCR amino acid sequence or a fragment thereof containing at least one epitope capable of being recognized by an anti-EPCR autoantibody, is a polypeptide such as that previously defined on describing the ELISA test for detecting and/or quantifying anti-EPCR autoantibodies. In a particular embodiment, this polypeptide is the so-called rhsEPCR (see Example), consisting of a fusion protein comprising the amino acid sequence of the soluble part of human EPCR (hsEPCR), the amino acid sequence corresponding to c-myc epitope, and a tail of histidines—the sequence of which is shown in SEQ ID NO: 3.

In another aspect, said kit is used to evaluate in vitro the risk and susceptibility of a subject to develop a pathology associated with the presence of high levels of anti-EPCR autoantibodies, selected from an autoimmune disease, a vascular disease and obstetric complications.

The following examples illustrate the invention.

### EXAMPLE 1

Use of Anti-EPCR Autoantibodies as Markers for the Risk and Susceptibility of a Subject to Develop Pathologies Related to the Presence of High Levels of Said Autoantibodies

### I. Materials and Methods

Patients

1. Patients with APLS and Controls

The study comprised a total of 43 patients [age 44±11 years (mean±standard deviation (SD)), 39 women and 4 males] diagnosed with antiphospholipid syndrome (APLS) according to international diagnostic criteria [Wilson W A, Gharavi A E, Koike T, Lockshin M D, Branch D W, Piette J C, Brey R, Derksen R, Harris E N, Hughes G R, Triplett D A, Khamashta M A. International consensus statement on preliminary classification criteria for definite antiphospholipid syndrome: report of an international workshop. Arthritis Rheum. 1999; 42:1309-11; Brandt J T, Barna L K, Triplett D A. Laboratory identification of lupus anticoagulants: results of the Second International Workshop for Identification of Lupus Anticoagulants. On behalf of the Subcommittee on Lupus Anticoagulants/Antiphospholipid Antibodies of the ISTH. Thromb Haemost. 1995; 74:1597-603] between February 1998 and March 2002. All patients were characterized by presenting anticoagulant lupus antibodies (ACL) and a personal history of venous thrombosis (n=17), arterial thrombosis (n=13, of which 4 referred acute myocardial infarction (AMI), 7 presented cerebrovascular thrombotic disease (CVTD), and 2 showed disease in other regions), or both [n=13, all presenting deep venous thrombosis plus CVTD (n=8), AMI (n=1), CVTD plus AMI (n=3) or arterial thrombosis in the mesenteric region (n=1)]. Twenty-seven of these patients were diagnosed with systemic lupus erythematosus (SLE). Serum samples were collected during the time in which ACL was positive, and at least 3 months after the last thrombotic episode. The samples were stored at −80° C. until processing for the detection of anti-EPCR autoantibodies.

The control group consisted of 43 healthy volunteers with no history of thrombosis or ACL. All patients and controls had given informed consent to participation in the study.

2. Women with Fetal Death and Controls

A paired case-control study of fetal death was carried out. A total of 87 women, aged 19 to 31 years (mean: 27 years), were included in the study between September 1996 and September 2002 due to a first episode of fetal death in the tenth week of amenorrhea, and occurring in their last pregnancy. The study excluded women with thrombotic antecedents, a history of chronic infectious diseases or some known systemic disease, diabetes mellitus or with antecedents of other types of gestational pathology (spontaneous miscarriage, eclampsia, restricted intrauterine fetal growth), as well as cases of fetal death due to some chromosomal anomaly affecting the karyotype, or morphological malformation of the fetus. Fetal death occurred during first pregnancy in 58 women, during second pregnancy in 21 women, and during third pregnancy in the remaining 8 women; in 75 women the event took place between weeks 10 and 22, while in the remaining 12 women fetal death occurred between weeks 22 and 36 (mean: 17 weeks).

A control group of 87 healthy mothers was established, grouped by age, number of pregnancies and the time elapsed from the last pregnancy; all satisfied the exclusion criteria applied to the group of women with fetal death. The controls were recruited concomitantly during the same time period, from women seen as outpatients in the Department of Gynecology of the same Hospital, for systematic medical examination.

The study was approved by the ethics committees of the inventors institution, and informed consent was obtained from all subjects. The inclusion of patients and controls, informed consent, and the collection of blood samples took place at least 6 months (range: 6-12 months) after fetal death. The blood samples were collected, processed and stored at −80° C., according to conventional procedures. The sampling protocols were identical in all cases and controls.

Expression of Recombinant Human Soluble EPCR

For the expression of human recombinant EPCR in soluble form (rhsEPCR), amplification was performed of the human soluble EPCR (hsEPCR) sequence, comprising the extracellular domain without its signal peptide or the transmembrane and intracellular domains (Entrez-Protein 21730830, residues 1-193, numbering corresponding to the mature form of the protein after processing of the signal peptide), via polymerase chain reaction (PCR) with the primers

SEQ ID NO: 1 and

SEQ ID NO: 2,

which added a ClaI restriction site and another NotI site at the 5′ and 3′ extremes, respectively, using cDNA from endothelial cells as template. These modifications allowed binding of the rhsEPCR sequence to the ClaI and NotI sites of plasmid pPICZαC (Stratagene, La Jolla, Calif.) following the secretion signal of factor α from Saccharomyces cerevisiae, permitting efficient secretion of many proteins into the extracellular medium from the interior of yeast cells.

The insert was cloned in reading phase with a c-myc epitope and a 6-histidine tag present in the pPICZαC vector. Due to the cloning process a serine residue and an isoleucine residue were added at the amino extreme of rhsEPCR, which is expressed fused to its carboxy-terminal end to a tail or tag containing the c-myc epitope and 6 histidines to facilitate purification and anchoring of rhsEPCR to the bottom of the microplate wells via an anti-c-myc monoclonal antibody. By direct sequencing, it was confirmed that the insert and vector sequences were correct. SEQ ID NO: 3 shows the sequence of rhsEPCR thus obtained, deduced from the DNA sequence, comprising the residues added by the cloning technique employed, the residues of the extracellular region of human rhsEPCR, the c-myc epitope, and the tag of 6 histidines.

With the previously prepared expression vector and after linearization of the latter with restriction enzyme PmeI, Pichia pastoris cells were transformed by means of a chemical method (Easy Comp, Invitrogen), yielding the integration, via homologous recombination, of the sequence encoding rhsEPCR in the methanol response endogenous promoter. The transformation product was cultured in presence of zeocine to select those colonies of P. pastoris transformed with the vector containing the rhsEPCR encoding sequence, which in turn contains the gene encoding resistance to zeocine. Briefly, the transformed yeasts were cultured in 4 ml of BMY medium [1% (w/v) of yeast extract, 2% (w/v) of peptone, potassium phosphate 100 mM (pH 6.0), 1.34% (w/v) of yeast nitrogen source with ammonium sulfate, 4×10-5% (w/v) of biotin] supplemented with 1% (v/v) of glycerol (BMGY), and incubated at 28-30° C. for about 18 hours with stirring. Cells were collected by centrifugation at 2000 g during 5 minutes at room temperature. The supernatant was discarded, and expression of rhsEPCR was induced with 1% methanol during 18 hours. To this effect, the cells were resuspended in 3 ml of BMY supplemented with 0.5% (w/v) of methanol, followed by incubation for 18 hours at approximately 28-30° C. under vigorous stirring. Following induction, the samples from the conditioned medium were loaded on 12% NuPAGE Bis-Tris gels (Invitrogen, Carlsbad, Calif.), and rhsEPCR was detected by Western Blot using the anti-myc monoclonal antibody (Invitrogen). For large scale production, selection was made of the colony that secreted the highest concentration of rhsEPCR. From the colonies selected on the basis of their high production of rhsEPCR, studies were made of colony methanol metabolism (rapid or slow metabolizer), thus allowing definition of the optimum expression conditions for the most adequate colony. Following optimization of the culture conditions and induction with methanol, the scale was increased for the production of large amounts of rhsEPCR.

Purification of Recombinant sEPCR

Since P. pastoris secretes very few proteins into the medium, a high percentage of the proteins found in the culture medium correspond to rhsEPCR—a fact that considerably simplified purification thereof. Briefly, rhsEPCR was purified from the supernatants of the yeast cultures by means of a triple-step purification process comprising metal affinity chromatography, anion exchange and gel filtration chromatography. To this effect, the supernatant of the culture was concentrated and dialyzed against sodium phosphate 100 mM, NaCl 10 mM, pH 7.6, followed by metal affinity chromatography in a 5-ml Hitrap column (Amersham Biosciences, Little Chalfont, United Kingdom) loaded with copper. The fraction that bound to the column was eluted with a buffer containing ethylenediaminetetraacetic acid (EDTA), and was dialyzed against Tris-HCl 20 mM (pH 7.6) without NaCl. Next, anion exchange chromatography was carried out in a Resource Q column (Amersham Biosciences), and elution was performed with a 0.0-300 mM gradient of NaCl in a volume equivalent to that of 20 columns. The eluted fractions containing rhsEPCR were pooled and concentrated by centrifugation-ultrafiltration, and then loaded on a Superdex 75-HR10/30 column (Amersham Biosciences) for gel filtration. The concentration of purified protein was determined using the BCA total protein test (Pierre, Rockford, Ill.) and standards of bovine serum albumin (BSA). For the detection of purified rhsEPCR, the samples were loaded on 12% NuPAGE Bis-Tris gels (Invitrogen, Carlsbad, Calif.), and electrophoresis was performed under reducing conditions followed by staining with Coomassie blue. One electrophoresis gel was subjected to electroblotting, and rhsEPCR was detected with anti-myc monoclonal antibody (Invitrogen). In order to estimate the molecular weight of rhsEPCR, use was made of a molecular weight standard included in each electrophoresis gel.

ELISA for the Determination of Anti-EPCR Autoantibodies in Serum or Plasma

Separate determinations were made of the levels of anti-EPCR autoantibodies corresponding to isotypes IgG, IgA or IgM, since these are the forms most frequently found in patients with autoimmune alterations, where antibodies targeted to some of the host structures are detected (autoantibodies).

In all three cases 96-well microplates (Costar, Acton, Mass., USA) were coated with 100 μl/well of anti-c-myc monoclonal antibody (Invitrogen, USA) at a concentration of 1.5 μg/ml in a solution of Na₂CO₃ (100 mmol/l), pH 9.6, overnight at a temperature of 4° C. This antibody is used as capture antibody, and is targeted to the added c-myc tag present in rhsEPCR. In this way, rhsEPCR is anchored to the well, preserving its extracellular epitopes. After washing with TB (Tris 20 mM, NaCl 150 mM, 0.05% of Tween-20, pH 7.4), the nonspecific binding sites were blocked with 3% (w/v) of BSA in TB at room temperature (RT) during 4.5 hours. Then, 100 μl/well of a solution containing 3 μg/ml of rhsEPCR in TB supplemented with 1% BSA (TB1) was added, followed by incubation during 2 hours at room temperature with gentle stirring. In parallel, blank wells were incubated with TB1 in the absence of rhsEPCR. After washing with TB, 100 μl of a 1:100 (plasma or serum) dilution of the sample in TB1 was added to each well, followed by incubation overnight at 4° C. The wells were then washed with TB, and the anti-EPCR autoantibodies remaining bound to the bottom of the wells were detected with murine anti-human IgA polyclonal antibody conjugated to peroxidase (Biotrend), murine anti-human IgM polyclonal antibody conjugated to peroxidase (Zymed), or murine anti-human IgG polyclonal antibody conjugated to alkaline phosphatase (Zymed). After a 2 hour incubation period at room temperature with gentle stirring, washing was performed.

To determine the levels of anti-EPCR autoantibodies of IgA or IgM isotype, 100 μl of a solution (0.4 mg/ml) of or-phenylendiamine (Kodak) was added, containing Na₂HPO₄ 0.07 M, sodium citrate 0.04 M and 0.02% (v/v) of H₂O₂, pH 5.0. After a development period of 5 and 8 minutes in the dark for IgA and IgM, respectively, 100 μl of H₂SO₄ was added to stop the reaction, and 5 minutes later readings were obtained of the absorbances at 492 nm in a microplate reader (iEMS REader, Labsystems, Finland).

In the plate used to assay anti-EPCR autoantibodies of IgG isotype, 100 μl of a solution (1 ng/ml) of 4-nitrophenyl phosphatase (Sigma) in diethanolamine 0.1 M, pH 10.3 was added. After 15 minutes, the reaction was stopped with 100 μl of NaOH 1 M, and the absorbance was recorded after color stabilization at 405 nm in the microplate reader (iEMS REader) . All samples were assayed at least twice in different tests.

To ensure that all absorbances measured in each plate corresponded to a linear range, construction was made, for each isotype, of a curve using serial dilutions of the sample whose absorbance was the highest recorded. To allow comparisons between plates, a sample was selected for testing in each plate (standard sample)—thus allowing introduction of a correction factor. The arbitrary units (AU) were defined as follows: for each patient sample (study sample) the specific absorbance was calculated substracting the absorbance of the blank wells and then multiplying by 1000 and by a correction factor corresponding to the ratio between the specific absorbance of the standard sample tested in a given plate (reference plate) and in the plate where the study sample was tested. The inter- and intra-test coefficients of variation (CV) were evaluated using 5 samples tested 5 times for the inter-test coefficient of variation (less than 5%), and three different times for calculating the inter-test coefficient of variation (less than 10%).

Generation of APC in Cultured Endothelial Cells

The cell line used was EA.hy926, a line of transformed human endothelial cells that have retained the capacity to express thrombomodulin and EPCR (Stearns-Kurosawa D J, Kurosawa S, Mollica J S, Ferrell G L, Esmon C T. The endothelial cell protein C receptor augments protein C activation by the thrombin-thrombomodulin complex. Proc Natl Acad Sci USA. 1996; 93:10212-6). 5×10⁴ cells/well were incubated in a 96-well plate with 0.02 U/ml of thrombin (0.17 nM) (ERL, Swansea, United Kingdom) and growing concentrations of PC (Baxter, Deerfield, Ill., USA) between 50 and 1000 nM in Tris 20 mM buffer, pH 7.4, supplemented with NaCl 150 mM, CaCl₂ 5 mM, MgCl₂ 0.6 mM, 1% BSA, 0.001% Tween-20 and 0.02% NaN₃. After 45 minutes at room temperature, lepirudin was added (Schering A G, Berlin, Germany) at an end concentration of 0.2 μmol/L, to inhibit thrombin; 3-4 minutes later, chromogenic substrate S-2366 was added (Chromogenix, Milan, Italy) at an end concentration of 0.4 mM with the purpose of monitoring its proteolysis by APC. The increase in absorbance at 405 nm was recorded kinetically with a microplate reader (iEMS REader, Labsystems, Finland). Curve data fitting to the Michaelis-Menten equation was carried out using the Enzfitter program (Biosoft, Cambridge, United Kingdom), which calculated the Km of PC activation under those conditions. Where necessary, 45 μg/ml of purified anti-EPCR autoantibodies from patients (see below) were added simultaneously with the thrombin and PC. Thus, the effect of the anti-EPCR autoantibodies upon PC activation could be analyzed.

Purification of Anti-EPCR Antibodies

1. Purification of IgM Antibodies

One-ml samples of serum containing anti-EPCR autoantibodies were diluted in phosphate buffered saline (PBS) (sodium phosphate 100 mM, NaCl 0.15 M, pH=7.4) and filtered through a filter of 0.45 μm pore size. The filtrate was applied to a HitTrap column activated by NHS HP (Amersham Biosciences), where murine anti-human IgM polyclonal antibody had been previously immobilized (Maruyama S, Kubagawa H, Cooper M D. Activation of human B cells and inhibition of their terminal differentiation by monoclonal anti-murine antibodies. J Immunol. 1985; 135:192-9). Human IgM was eluted with 5 ml of glycine 0.1 M, pH 2.5 and collected in 100 μl of Tris 1 M, pH 9.0. The fraction containing human IgM was concentrated and dialyzed against TB supplemented with CaCl₂ 5 mM and MgCl₂ 0.6 mM, pH 7.4.

2. Purification of IgA Antibodies

One-ml samples of serum were diluted in PBS and manually applied to a jacaline column (Pierce). The adsorbed fraction was eluted with 2 ml of mellibiose 0.1 M in PBS and then dialyzed against PBS. Since posterior purification steps were required, the samples were applied to a HiTrap Protein G HP affinity column (Amersham Biosciences) to remove the contaminating IgG. The unbound product containing the IgA fraction was dialyzed against KH₂PO₄ 50 mM, pH 7.0, and finally applied to a HiTrap Blue HP affinity column (Amersham Pharmacia Biotech) to remove albumin. The unbound material containing the purified IgA fraction was then collected and dialyzed against TB buffer supplemented with CaCl₂ 5 mM and MgCl₂ 0.6 mM, pH 7.4.

3. Purification of IgG Antibodies

One-ml samples of serum containing anti-EPCR autoantibodies were diluted in phosphate buffered saline (PBS) (sodium phosphate 100 mM, NaCl 0.15 M, pH=7.4) and filtered through a filter of 0.45 μm pore size. The filtrate was applied to a HitTrap Protein G HP column (Amersham Biosciences). The human IgG was eluted with 5 ml of glycine 0.1 M, pH 2.5 and collected in 100 μl of Tris 1 M, pH 9.0. The fraction containing the human IgM was concentrated and dialyzed against TB supplemented with CaCl₂ 5 mM and MgCl₂ 0.6 mM, pH 7.4.

Preparation of a rhsEPCR Affinity Column

rhsEPCR (2 mg in 3 ml of NaHCO₃ 100 mM, pH 8.5) was bound to a HitTrap NHS-activated HP affinity column (Amersham Biosciences) following the manufacturer's instructions. Once the reaction had been stopped with glycine 0.1 M, the rhsEPCR column was thoroughly washed with NaCl 2 M. This way the rhsEPCR column was able to bind PC in TBS, pH 7.4, supplemented with CaCl₂ 20 mM and MgCl₂ 0.6 mM. The PC could be eluted from the column with TBS supplemented with EDTA (data not shown). Since the rhsEPCR bound to the column maintained its capacity to bind PC, it could surely retain the native conformation and epitopes recognized by the autoantibodies. Consequently, the column thus prepared was adequate for eliminating anti-EPCR autoantibodies from a sample of serum or plasma.

Statistical Methods

In the study of APLS cases and controls, the comparison between patients (cases) and controls of the frequency of high levels of anti-EPCR IgM, IgA and IgG antibodies was carried out using the chi-squared test. The odds ratio (OR) (Martínez-González M A, of Irala-Estevez J & Guillén Grima F, (1999), [symbol] Qué es una odds ratio?, Medicina Clínica, 112, 11:416-422) and the 95% confidence interval (95% CI) were calculated as a measure of the association between APLS and anti-EPCR autoantibodies.

In the paired case-control study of fetal death, the comparison between cases and controls for continuous variables and by categories was carried out with the t-test for paired samples and with the McNemar test, respectively. The association between the levels of anti-EPCR autoantibodies corresponding to isotypes IgG and IgM with ACL and with anti-cardiolipin antibodies of IgM isotype was assessed based on the correlation coefficients for continuous variables and the Mann-Whitney test for variables by categories.

To evaluate the risk of fetal death associated with high levels of anti-EPCR autoantibodies of IgG and IgM isotypes, multiple regression analysis was used with case-control pairs. The principal independent variables were the levels of anti-EPCR autoantibodies corresponding to isotypes IgG and IgM by categories, according to the distribution of these immunoglobulins in controls. Different cutoff points were used to determine the levels associated to a higher risk. Uni- and multivariate analyses were carried out, fitting for known fetal death risk factors. It was not possible to include factors V Leiden (FVL) and ACL in the complete model; consequently, two models were considered for testing the effect of anti-EPCR autoantibodies:

(1) simultaneously introducing the levels of anti-EPCR autoantibodies corresponding to isotypes IgM and IgG, anti-cardiolipin antibody of IgM isotype, ACL and prothrombin G20210A; and

(2) identical to model 1, but adjusting for the presence/absence of FVL, instead of ACL.

Model (1) was used to evaluate the hypothesis that anti-cardiolipin antibodies and ACL are markers, rather than etiological factors, indicating a prothrombotic status caused by anti-EPCR autoantibodies. All calculations were made using SPSS, version 10.0 statistical package (SPSS Inc.).

### II. Results

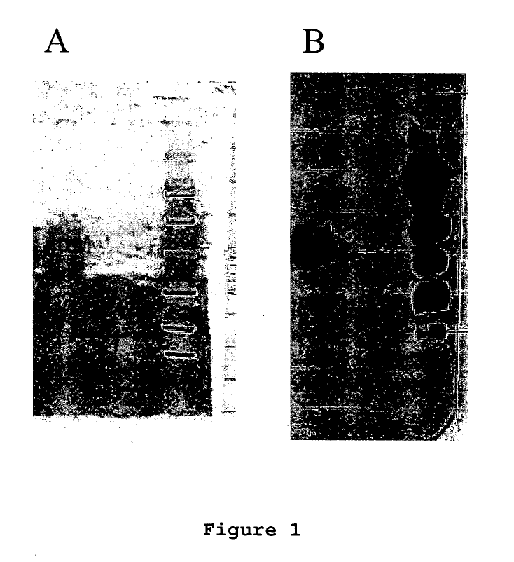

With the aim of investigating the presence of anti-EPCR autoantibodies in plasma and serum, rhsEPCR was first produced using the expression system of the yeast P. pastoris. Based on the described protocol, it was possible to purify more than 5 mg of rhsEPCR from a P. pastoris culture. Using electrophoresis in polyacrylamide with sodium dodecylsulfate (SDS-PAGE) gel and Western blot analysis with anti-myc monoclonal antibody, rhsEPCR appeared as a single and slightly heterogeneous band, reflecting the different degrees of glycosylation, as previously reported (Fukudome K, Kurosawa S, Stearns-Kurosawa D J, He X, Rezaie A R, Esmon C T. The endothelial cell protein C receptor. Cell surface expression and direct ligand binding by the soluble receptor. J Biol Chem. 1996; 271:17491-8) [see FIG. 1].

The rhsEPCR was able to inhibit the anticoagulant activity of APC in the context of a coagulation test, as has been previously described (Regan L M, Stearns-Kurosawa D J, Kurosawa S, Mollica J, Fukudome K, Esmon C T. The endothelial cell protein C receptor. Inhibition of activated protein C anticoagulant function without modulation of reaction with proteinase inhibitors. J Biol Chem. 1996; 271:17499-503) (data not shown). In addition to binding PC with the expected affinity (see below), the activation of PC by thrombin on the surface of endothelial cells was characterized by a Km of 51±10 nM. The activation decreased considerably in the presence of rhsEPCR 2 μM (Km=1000 nM approximately), implying a Ki of 70 nM approximately, and suggesting that rhsEPCR binds PC with similar efficacy to that of native EPCR, as has been previously reported (Fukudome K, Kurosawa S, Stearns-Kurosawa D J, He X, Rezaie A R, Esmon C T. The endothelial cell protein C receptor. Cell surface expression and direct ligand binding by the soluble receptor. J Biol Chem. 1996; 271:17491-8; Regan L M, Stearns-Kurosawa D J, Kurosawa S, Mollica J, Fukudome K, Esmon C T. The endothelial cell protein C receptor. Inhibition of activated protein C anticoagulant function without modulation of reaction with proteinase inhibitors. J Biol Chem. 1996; 271:17499-503) . Such evidences strongly suggest the correct rhsEPCR activity and conformation—thus allowing its use for the detection of antibodies against human EPCR.

Anti-EPCR Autoantibodies in Patients with APLS

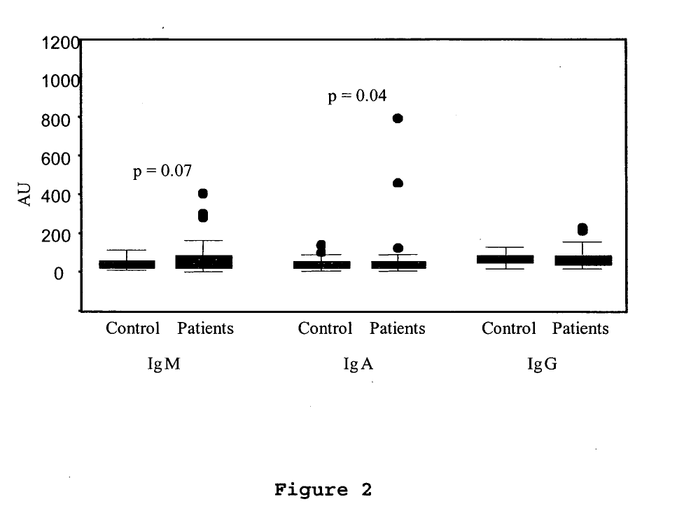

Taking high levels to represent those above percentile 97 for each control group, high levels of anti-EPCR autoantibody corresponding to isotypes IgM, IgA or IgG were associated with APLS [OR=4.47; 95% CI: 1.15-17.40] (See Table 1). Extremely high levels of anti-EPCR autoantibodies (see FIG. 2) were only detected in subjects diagnosed with APLS: three patients showed very high levels of anti-EPCR autoantibodies of IgM isotype (patient A=407 AU, patient B=301 AU, and patient C=293 AU), two patients with APLS presented very high levels of anti-EPCR autoantibodies of IgA isotype (patient D=795 AU and patient B=475 AU, who also showed high levels of anti-EPCR autoantibodies of IgM isotype), and two patients presented high levels of anti-EPCR autoantibodies of IgG isotype (patient E=230 AU and patient F=220 AU). The 6 patients were women with a prior history of thrombosis—this being one of the selection criteria (stroke in patients A, C, D and F; cardiovascular disease in patient E; venous thrombosis in patients A, B, D and F). The most interesting observation is the fact that all the women presenting anti-EPCR autoantibodies of isotypes IgM and IgA (except patient B, who proved non-evaluable) suffered multiple episodes of fetal death.

In view of this finding, the analysis was directed towards the possible association between anti-EPCR autoantibodies and fetal death.

TABLE 1
OR of APLS associated with anti - EPCR antibodies

| Anti-EPCR | | | |
|---|---|---|---|
| autoantibodies | APLS | Control | |
| (percentile > 97%) | (n = 43) | (n = 43) | OR (95% CI) |
| IgG | | 1 | 3.10 (0.30-31.50) |
| IgM | 6 | 1 | 6.80 (0.80-59) |
| IgA | 3 | 1 | 3.10 (0.30-31.50) |
| IgG + IgM + IgA | 11 | 3 | 4.47 (1.15-17.4) |

Biochemical Characterization of Anti-EPCR Antibodies

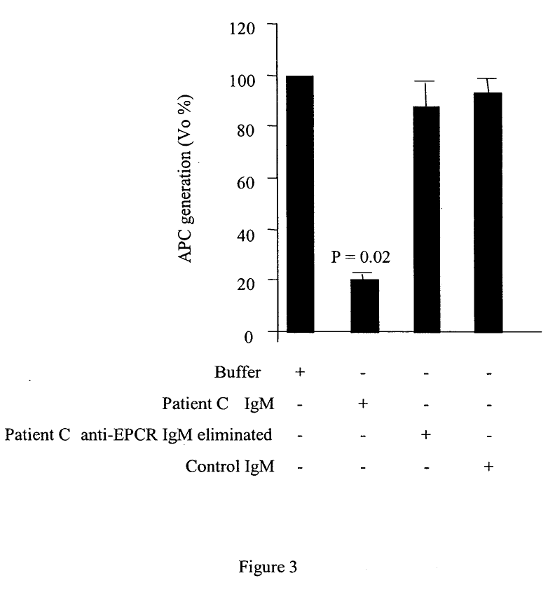

The fractions of anti-EPCR autoantibodies of isotype IgM, IgA and IgG from patients with extremely high levels were purified from 1 ml of serum. The fraction of anti-EPCR autoantibodies of IgM isotype in patient C was able to reduce the generation of APC by cultured endothelial cells in the presence of thrombin (20% of the residual capacity of PC activation, p=0.02). The inhibitory effect was dose-dependent. In order to demonstrate that this effect of the fraction of anti-EPCR autoantibodies of IgM isotype in the patient with APLS was due to a specific antibody against EPCR, the sample was completely deprived of specific anti-EPCR autoantibody, loading it in an affinity column where rhsEPCR was immobilized. The fraction thus obtained lost its inhibitory action upon APC generation (87.6% of PC generation), which implies that the agent responsible for the phenomenon must have been a specific anti-EPCR autoantibody. None of the other fractions purified from patients with APLS were able to modify the capacity of endothelial cells to generate APC (FIG. 3).

Anti-EPCR Autoantibodies in Women with Fetal Death

The frequencies of the risk factors previously related to fetal death and of the anti-EPCR antibodies in the group of patients and controls are shown in Table 2.

TABLE 2
Univariate ORs and their confidence intervals for fetal death
associated to the different variables studied

| | Fetal | | | | |
|---|---|---|---|---|---|
| | death | Controls | | | |
| | (n = 87) | (n = 87) | Paired OR | 95% CI | p |
| Anti-EPCR IgM | 16 | 3 | 14.0 | (1.8-106.4) | 0.01 |
| Anti-EPCR IgG | 13 | 4 | 4.3 | (1.2-15.2) | 0.02 |
| Factor V Leiden | 6 | 1 | 6.0 | (0.7-49.8) | 0.1 |
| Prothrombin | 3 | 1 | 3.0 | (0.3-28.8) | 0.34 |
| G20210A | | | | | |
| ACL | 7 | 1 | 7.0 | (0.9-56.9) | 0.07 |
| Anticardiolipin | 9 | 2 | 5.0 | (1.1-22.8) | 0.04 |
| IgM | | | | | |
| Anticardiolipin | 1 | 0 | — | — | — |
| IgG | | | | | |

Percentile of 95% of the levels of anti-EPCR autoantibody of IgM isotype in the control group was 99 AU. Of the 87 patients, 16 (18%) presented values that exceeded this cutoff point, versus three subjects in the control group (n=87). The OR not adjusted for fetal death in patients with levels of anti-EPCR autoantibodies of IgM isotype in excess of percentile 95 compared with those presenting a lower value was 14 (95% confidence interval (CI): 1.8-106.4). When the cutoff point was established at 90% percentile (83 AU), the OR was 5.2 (95% CI: 1.8-15.3).

Percentile 95 of the levels of anti-EPCR autoantibody of IgG isotype in the control group was 94 AU. Of the 87 patients, 13 (15%) showed values that exceeded this cutoff point, versus 4 subjects in the control group. The OR not adjusted for fetal death in patients with anti-EPCR autoantibodies of IgG isotype in excess of 95% percentile was 4.3 (95% CI: 1.2-15.2). When the cutoff point was established at a 90% (88.4 AU), the OR was 2.3 (95% CI: 0.9-5.6).

In addition, a multivariate analysis has been made adjusting for potential confounding factors. As commented above, it was not possible to include FVL and ACL in the same multivariate model—as a result of which two different models were considered: Model (1), adjusted for antiphospholipid antibodies (i.e., ACL and anticardiolipin antibodies) and prothrombin G20210A; and Model (2), including FVL but not ACL. The OR associated with anti-EPCR autoantibodies of IgM isotype in excess of percentile 95 in Model (1) was 23.1 (95% CI: 2-266.3) while in Model (2) the value was 31.0 (95% CI: 2-384.3). The OR associated with anti-EPCR autoantibodies of IgG isotype in excess of percentile 95 in Model (1) was 6.8 (95% CI: 1.2-38.4). According to Model (2), which includes factor V of Leiden instead of ACL, the OR associated with anti-EPCR autoantibodies of IgG isotype in excess of percentile 95 was 11.0 (95% CI: 1.6-73.5). The results are shown in Table 3.

TABLE 3
Multivariate ORs and their 95% confidence intervals for fetal death
associated to high levels of anti-EPCR autoantibodies

| | Model 1 | | | Model 2 | | |
|---|---|---|---|---|---|---|
| Anti-EPCR | Paired | | | Paired | | |
| autoantibodies | OD | 95% CI | P* | OD | 95% CI | p* |
| Anti-EPCR | 23.0 | 2.0-266.3 | 0.012 | 31.0 | 2.0-384.3 | 0.007 |
| IgM | | | | | | |
| Anti-EPCR | 6.8 | 1.2-38.4 | 0.029 | 11.0 | 1.6-73.5 | 0.013 |
| IgG | | | | | | |

These results indicate that anti-EPCR autoantibodies of isotypes IgM and IgG are independent risk factors for fetal death. However, high levels of IgA were not significantly associated to fetal death in this group of women.

### III. Discussion

A method (specifically, an ELISA test) has been implemented that allows detection of the presence of autoantibodies against human EPCR. Using this system, a study has been made of a group of patients with APLS characterized by thrombosis and ACL, demonstrating (for the first time in human pathology) the presence of specific anti-EPCR autoantibodies of isotypes IgM, IgG and IgA. The study centered on the subgroup of patients with APLS and ACL because they have been associated with an increased risk of thrombosis; consequently, these subjects would be likely to present autoantibodies directly related to the clinical manifestations. In fact, many patients were found to have very high levels of anti-EPCR autoantibodies.

These autoantibodies could provide an explanation for the thrombosis and miscarriages seen in patients with APLS. Firstly, EPCR is a molecule expressed on the endothelium of large blood vessels and trophoblast. IgM and IgG immunoglobulins can bind and activate complement; if these antibodies are targeted to EPCR, they could activate complement on the endothelium and damage the latter—thus promoting thrombosis at this level. Secondly, it has been shown that the IgM fraction of a patient with high levels of anti-EPCR autoantibodies of IgM isotype can greatly reduce the generation of APC by endothelial cells in the presence of thrombin. This inhibitory effect disappears after specifically eliminating IgM targeted to EPCR by jointly passing the IgM fraction through an EPCR affinity column—which means that the inhibitory effect is due to an anti-EPCR autoantibody of IgM isotype. This antibody would probably result in low levels of APC in vivo—a situation in itself constituting a strong risk factor for thrombosis.

On selecting the patients according to the criterion of venous and/or arterial thrombosis, it was not possible to evaluate the risk of thrombosis associated with anti-EPCR autoantibodies. In contrast, increased levels of anti-EPCR autoantibodies, particularly of IgM isotype, were detected in women with a prior history of fetal death versus women without such antecedents. In view of these results in the pilot study, the decision was made to conduct a paired case-control study to evaluate the risk of a first episode of unexplainable fetal death in a general population of women associated with the presence of anti-EPCR autoantibodies. It was seen that high levels of anti-EPCR autoantibodies of IgM isotype (defined as a value in excess of percentile 95 of the value distribution in control subjects) constitute a strong risk factor for first episodes of fetal death, with a relative risk of 23 or 31, compared with lower levels. High levels of anti-EPCR autoantibodies of IgG isotype also constituted a strong risk factor, though less so than in the case of IgM isotype, with a relative risk of 7 or 11—depending on the mathematical model used. In the univariate analysis, ACL and anticardiolipin antibody of IgM isotype were associated to an increased risk of fetal death, though this association was attenuated in the multivariate model—possibly because the information afforded by classical antiphospholipid antibodies is attributable to the associated anti-EPCR autoantibodies, which could represent an etiological factor of fetal death rather than a simple risk marker. Likewise, a study was made of the presence of FVL and prothrombin G20210A, which have recently been associated with an increased risk of late fetal death—an increased risk being identified in both the univariate and multivariate analyses in association to such polymorphism. The risk was not statistically significant, however, probably because of the number of patients included in the study.

In conclusion, this study for the first time demonstrates the presence of anti-EPCR autoantibodies in patients with APLS and thrombosis. The presence of anti-EPCR autoantibodies of IgM and IgG isotypes increases the risk of a first episode of fetal death. These autoantibodies may intrinsically contribute to thrombosis and to fetal death in patients with APLS and in the general population.

### EXAMPLE 2

### Detection of Anti-EPCR Autoantibodies in Women with Myocardial Infarction

Study group: 142 women (aged 39±5 years, mean±standard deviation) with myocardial infarction, and 142 healthy women (aged 39±5 years), matched by age and geographical origin. A study was made of the classical myocardial infarction risk factors (hypertension, hypercholesterolemia, diabetes, smoking and oral contraceptives). Assays were made of anti-EPCR autoantibodies IgG, IgM and IgA in samples of plasma, following the ELISA test protocol described under “Materials and Methods” (Example 1).
Results: High levels of anti-EPCR autoantibodies, defined by values in excess of percentile 93 of the distribution of the levels of anti-EPCR antibodies in the control group, were associated to an increased risk of myocardial infarction. In the multivariate analysis, high levels of anti-EPCR antibodies were associated to an adjusted odds ratio (OR) of 3.5, with a 95% confidence interval (95% CI) of 1.4-8.9 for IgA, while in the case of IgM the figures were OR=3.0; 95% CI: 1.2-7.5.
Conclusion: High levels of anti-EPCR autoantibodies constitute an independent risk factor for myocardial infarction in women.

1. A method to evaluate the presence of high levels of autoantibodies against endothelial protein C (PC)/activated PC receptor (EPCR) in a sample, said method comprising quantifying in vitro autoantibodies against EPCR in said sample from a subject.

2. Method according to claim 1, wherein said presence of high levels of autoantibodies against EPCR being related to a pathology selected from the group consisting of autoimmune disease, vascular disease and obstetric complications.

3. Method according to claim 2, wherein said autoimmune disease is selected from the group consisting of antiphospholipid syndrome, systemic lupus erythematosus, rheumatoid arthritis and autoimmune vasculitis.

4. Method according to claim 2, wherein said vascular disease is selected from the group consisting of arterial vascular disease, venous vascular disease and thrombosis of the microcirculation.

5. Method according to claim 4, wherein said vascular disease is selected from the group consisting of myocardial infarction, cerebral stroke, a transient cerebrovascular accident, limb ischemia, atherosclerosis, aneurysm, thrombosis, superficial venous thrombosis, deep venous thrombosis, and pulmonary embolism.

6. Method according to claim 2, wherein said obstetric complication is selected from the group consisting of miscarriage, fetal death, premature birth, delayed intrauterine growth, eclampsia and pre-eclampsia.

7. Method according to claim 1, wherein said sample comprises serum or plasma.

8. Method according to claim 1, wherein said subject is human.

9. Method according to claim 1, wherein quantification of autoantibodies against EPCR is carried out by means of an immunoassay coupled to a marker.

10. Method according to claim 1, wherein quantification of autoantibodies against EPCR is determined using an ELISA test, said test comprising:
a) immobilizing an immobilizable polypeptide comprising the EPCR amino acid sequence or a fragment thereof containing at least one epitope that can be recognized by an anti-EPCR autoantibody on a solid support;
b) incubating the immobilized polypeptide with the sample obtained from the subject for sufficient time to allow binding of the anti-EPCR autoantibodies to the immobilized polypeptide, and the formation of polypeptide-anti-EPCR autoantibody complexes;
c) removing excess sample not bound to the immobilized polypeptide; and
d) incubating the polypeptide-anti-EPCR autoantibody complexes with a second antibody conjugated to an enzyme, where the second antibody is able to bind to the anti-EPCR autoantibodies.

11. Method according to claim 10, wherein said immobilizable polypeptide is selected from the group consisting of:
a) a polypeptide comprising the sequence of amino acids of full length EPCR; and
b) a polypeptide comprising the sequence of amino acids of a fragment of EPCR containing at least one epitope capable of being recognized by an anti-EPCR autoantibody.

12. Method according to claim 11, wherein said immobilizable polypeptide comprises a fusion protein comprising:
a) a region A comprising a first polypeptide containing the EPCR amino acid sequence or a fragment thereof containing at least one epitope capable of being recognized by an anti-EPCR autoantibody; and
b) a region B comprising a second polypeptide comprising a sequence of amino acids of use for isolating or purifying the mentioned fusion protein, and/or a sequence of amino acids of use for anchoring the mentioned fusion protein to a solid support.

13. Method according to claim 12, said region B is bound to the amino terminal extreme of region A.

14. Method according to claim 12, wherein said region B is bound to the carboxyl terminal extreme of region A.

15. Method according to claim 12, wherein said region A comprises the amino acid sequence of the soluble part of human EPCR.

16. Method according to claim 12, wherein the amino acid sequence of use for isolating or purifying the mentioned fusion protein, and/or an amino acid sequence of use for anchoring said fusion protein to a solid support present in region B, comprises a sequence selected from the group consisting of Arg-tag, His-tag, FLAG-tag, Strep-tag, an epitope capable of being recognized by antibody, SBP-tag, S-tag, calmodulin binding peptide, cellulose binding domain, chitin binding domain, glutathione S-transferase-tag, maltose binding protein, NusA, TrxA, DsbA, Avi-tag, Ala-His-Gly-His-Arg-Pro (SEQ ID NO: 4) (2, 4, and 8 copies), Pro-Ile-His-Asp-His-Asp-His-Pro-His-Leu-Val-Ile-His-Ser (SEQ ID NO: 5), Gly-Met-Thr-Cys-X-X-Cys (SEQ ID NO: 6) (6 repetitions), □-galactosidase and VSV-glycoprotein.

17. Method according to claim 12, wherein region B comprises a polypeptide comprising a c-myc epitope capable of being recognized by an anti-c-myc antibody and a tail of histidines (His-tag).

18. Method according to claim 12, wherein said immobilizable polypeptide is a fusion protein comprising the sequence of amino acids of the soluble part of human EPCR, the sequence of amino acids corresponding to c-myc epitope and a tail of histidines (His-tag).

19. Method according to claim 12, wherein said immobilizable polypeptide is a fusion protein comprising SEQ ID NO: 3.

20. Method according to claim 10, wherein said second antibody is an immunoglobulin isotype-specific antibody originating from a species different to that of the subject whose sample is being tested.

21. Method according to claim 20, wherein said immunoglobulin isotype-specific antibody is selected from the group consisting of an anti-human IgG antibody, an anti-human IgM antibody, an anti-human IgA antibody, and their mixtures.

22. Method according to claim 20, wherein said second antibody is conjugated to an enzyme selected from peroxidase or alkaline phosphatase.

23. Method according to claim 1, further comprising comparing quantified anti-EPCR autoantibody levels in the sample to normal levels of anti-EPCR autoantibody levels.

24. A method according to claim 1, wherein the variation in the levels of anti-EPCR autoantibodies are quantified over a given time period.

25. Method according to claim 24, wherein said sample originates from a subject previously diagnosed with an autoimmune or vascular disease, or who has suffered an obstetric complication, and is subject to therapeutic treatment.

26.-33. (canceled)

34. A kit for in vitro evaluation of the presence of high levels of autoantibodies against EPCR in a sample, said kit comprising an immobilizable polypeptide that comprises the EPCR amino acid sequence or a fragment thereof containing at least one epitope capable of being recognized by an anti-EPCR autoantibody.

35. The kit according to claim 34, wherein said immobilizable polypeptide comprises a fusion protein comprising:
i) a region A comprising a first polypeptide containing the EPCR amino acid sequence or a fragment thereof containing at least one epitope capable of being recognized by an anti-EPCR autoantibody; and
ii) a region B comprising a second polypeptide comprising an amino acid sequence of use for isolating or purifying the mentioned fusion protein, and/or an amino acid sequence of use for anchoring the mentioned fusion protein to a solid support.

36. The kit according to claim 35, wherein said region A comprises the amino acid sequence of the soluble part of human EPCR.

37. The kit according to claim 35, wherein said immobilizable polypeptide is a fusion protein comprising the amino acid sequence of the soluble part of human EPCR, the amino acid sequence corresponding to c-myc epitope and a tail of histidines (His-tag).

38. The kit according to claim 35, wherein said immobilizable polypeptide is a fusion protein comprising SEQ ID NO: 3.


SEQUENCE LISTING (6 sequences)

SEQ ID NO: 1 - DNA, 36 bp, Artificial Sequence
  Synthetic Construct
agcttggcatatcgattagccaagacgcctcagatg

SEQ ID NO: 2 - DNA, 36 bp, Artificial Sequence
  Synthetic Construct
tattctatgcggccgccgaagtgtaggagcggcttg

SEQ ID NO: 3 - protein, 222 aa, Artificial Sequence
  Synthetic Construct
SISQDASDGLQRLHMLQISYFRDPYHVWYQGNASLGGHLTHVLEGPDTNTTIIQLQPLQEPESWARTQSGLQSYLLQFHGLVRLVHQERTLAFPLTIRCFLGCELPPEGSRAHVFFEVAVNGSSFVSFRPERALWQADTQVTSGVVTFTLQQLNAYNRTRYELREFLEDTCVQYVQKHISAENTKGSQTSRSYTSAAASFLEQKLISEEDLNSAVDHHHHHH

SEQ ID NO: 4 - protein, 6 aa, Artificial Sequence
  Synthetic Construct
AHGHRP

SEQ ID NO: 5 - protein, 14 aa, Artificial Sequence
  Synthetic Construct
PIHDHDHPHLVIHS

SEQ ID NO: 6 - protein, 7 aa, Artificial Sequence
  Synthetic Construct
  MISC_FEATURE (5)..(6): X may be any essential amino acid
GMTCXXC